(19) United States
(12) Patent Application Publication
(10) Pub. No.: US 2010/0113958 A1
(43) Pub. Date: May 6, 2010

(54) Active Implantable Medical Device Integrating Spirometric Means for Diagnosing Lung Diseases

(76) Inventor: Cyrille Casset, Saint Selve (FR)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/614,087

(22) Filed: Nov. 6, 2009

(30) Foreign Application Priority Data
Nov. 6, 2008 (FR) 08/06199

(51) Int. Cl. A61B 5/08

(52) U.S. Cl. 600/538

(57) ABSTRACT

An active implantable medical device, integrating a spirometric function for the diagnosis of lung diseases is disclosed. The active implantable medical device measures the respiratory activity of a patient to collect a transthoracic impedance signal according to changes in lung volume. The active implantable medical device comprises a spirometer function produces, from the transthoracic impedance signal collected over a respiratory cycle, a characteristic curve that couples of flow values (dV/dt) as a function of pulmonary volume (V) that represents a spirometric characteristic (S) of a patient. An spirometric analysis is performed to deduce from the spirometric characteristic (S) at least one parameter of the patient's pulmonary status and to produce a diagnostic indicator based on the comparison of the at least one parameter with a predetermined reference value.

The present invention claims the benefit of and priority to French Application No. 08/06199 entitled “Active Implantable Medical Device integrating Spirometric Means For Diagnosing Lung Diseases” and filed on Nov. 6, 2008, which is incorporated herein by reference.

### FIELD

The present invention relates to the diagnosis of respiratory disorders in patients implanted with an “active implantable medical device” prosthesis, such as a device as defined by Directive 90/385/EEC of 20 Jun. 1990 Council of the European Communities, including an implantable cardiac device able to deliver at the heart electrical impulses for stimulation, re-synchronization, and cardioversion/defibrillation in case of a rhythm disorder detected by the device.

### BACKGROUND

It is known to provide a cardiac implant device with means for measuring respiratory activity of a patient implanted with the cardiac implant device. Trans-thoracic impedance signals are measured between two electrodes positioned in the patient's chest, or between the case of the device and a distant electrode, for example, a pacing electrode. The impedance is measured by injecting a current of several hundred microamperes, at a few Hertz, and measuring the responsive voltage between the electrodes. The variations of the instantaneous impedance measured according to this technique are representative of and reproduce the variations of the thoracic volume, with a minimum impedance being detected when the lungs are filled with air during an inspiratory phase, and an increasing impedance being detected during an expiratory phase.

The impedance signal is then used to produce an indication representative of a minute ventilation (volume x respiratory rate) after averaging over several tens of respiratory cycles. The minute ventilation signal is a good indicator of current metabolic needs of the patient, and is particularly useful for controlling the frequency of a cardiac pacemaker, for example, to increase or decrease the pacing frequency in case of varying metabolic needs.

The U.S. Patent application US 2006/0020295 A1 describes a device that characterizes a curve describing minute ventilation of a patient in the time domain in connection with the patient's activity, particularly from the peak-to-peak amplitude (tidal volume) and the respiratory rate.

The U.S. Pat. No. 7,329,226 B1 describes a device of a type similar to the one above that analyzes ventilatory patterns in distinguishing phases including inspiratory and expiratory phases that alternate over time.

The trans-thoracic impedance signal can also be used to detect and diagnose respiratory diseases, for example, apnea or hypopnea in the context of search for a syndrome of sleep apnea (SSA). Variations in the respiratory rate are analyzed to detect the occurrence of an apnea or a hypopnea and assess recurrences of them, which is more or less important, over an extended period of several hours (e.g., during a phase of sleep), even several days to assess the evolution of the syndrome. European Patent EP 0 970 713 A 1 and its counterpart U.S. Pat. No. 6,547,507 describes a device provided with means of diagnosis of SSA from a transthoracic impedance signal. Other diseases, such as the “Cheyne-Stokes” syndrome, which is a characteristic of patients with heart failure, can be detected by analyzing, with a monitoring of the transthoracic impedance signal, how the respiratory activity varies over several successive respiratory cycles. This technique is described, for example, in European Patent EP 1 295 623 and its counterpart U.S. Pat. No. 6,830,548, both assigned to the assignee of the present application, ELA Medical.

According to the present invention, the trans-thoracic impedance signal is used, not only for the subservience of a pacemaker or for the search of respiratory syndromes appearing over several respiratory cycles, but also for obtaining spirometric data, which may be useful to diagnose a lung disease.

Unlike known devices such as those cited above that seek to characterize the evolution of a ventilation curve in the time domain, a spirometric analysis is based on a curve plotted from representative data of the instant volumes and airflows (airflow rates) during a given respiratory cycle, said respiratory cycle being in principle a single, isolated cycleduring which the patient is asked to breathe in and out to his or her fullest.

The spirometric analysis is in itself well known and is performed by asking the patient to make a deep inspiration and a deep expiry in a specific device (e.g., spirometer) providing real-time measure of both inspired and expired air speed and volume during the test. The result can be represented in the form of a characteristic flow/volume (hereinafter referred to as “characteristic” or “curve”), and a practitioner can draw useful information with a visual examination of the morphology of the curve. Note that unlike the techniques mentioned above based on ventilation signal analysis, the time domain analysis is unnecessary in a spirometric analysis.

### OBJECTS AND SUMMARY

It is therefore an object of the present invention to provide a spirometric analysis for a patient implanted with a prosthesis equipped with means for measurement of transthoracic impedance (typically, circuits and logic for assessing minute ventilation), without use of a spirometer or any additional external equipment. The absence of use of any external device simplifies the data collection for a spirometric analysis, and the analysis can be performed in any circumstances and in any place, and even in an automatic manner.

It is another object of the present invention to perform spirometric measurement autonomously, without calibration or prior need for an external reference. Data from a spirometric measurement is stored in the memory of the implantable device and a regular or permanent monitoring of a patient's status using the collected data is performed. Therefore, it is possible to inform a physician during a routine visit or even without waiting for a visit, and to alert the patient or in an urgent case to call for a physician's attention to change one or more of the therapies delivered to the patient. The latter would include changing pacing and resynchronization therapies applied by the implanted device.

The value of such monitoring is even more desirable, considering the population of patients with heart failure, as the behaviour of the respiratory system is strongly impacted by cardiac decompensation due to a degradation of a patient's cardiac function. One of the main processes involves elevated blood pressure in lungs which have the effect of filling the lungs with water to resist the elevated blood pressure—the lung replaces air with water in order to increase the pressure side lungs to oppose the blood pressure side. This process has the effect of reducing the lung capacity of the patient making breathing more difficult and worsening oxygenation of the blood. The increase in the frequency of this process and the decrease in the respiratory volume may result in a premature heart failure. Conversely, a sustainable improvement in cardiac status of a patient has the opposite effect, and it may be visible on the patient's ventilatory signal. The population of patients suffering from heart failure is therefore a prime target for analysis and tracking of information on pulmonary ventilation from a spirometric analysis.

Beyond cardiac pacing, a spirometric analysis allows early detection of a chronic obstructive pulmonary disease (COPD). COPD is a serious illness, and is a major cause of death, with a frequent co-morbidity in patients with heart failure. The epidemiological studies suggest a strong association between the presence of COPD and the presence of ischemic heart disease, which is a major cause of heart failure. These irreversible disorders require continuous monitoring of the status of the patients, all the more necessary for they present double evolutions: slow and continuous, or rapid by sudden crises of ventilatory decompensation.

Advantageously, the present invention provides means for monitoring the lung status of a patient with COPD, with precision and without medical intervention. More advantageously, continuous monitoring of the status of a patient allows for a quick response by a physician in case of a significant pathology evolution. For example, a “home monitoring” technique enables a remote alert and transmission of the collected data at an opportune moment, thereby reducing the time for proper diagnosis and treatment upon the detection of a deterioration of the patient management while the patient is unaware of the seriousness of his or her condition.

To that purpose, the present invention provides an active implantable medical device, comprising means for measuring the respiratory activity of a patient and collecting a transthoracic impedance signal according to changes in instantaneous lung volume. In a manner characteristic of the present subject matter, the device further comprises spirometric means for producing, from the transthoracic impedance signal collected during at least one respiratory cycle, correlation between the instantaneous lung volume and the derivative of said lung volume, for further producing, from the obtained correlation, a spirometric characteristic defined in a space flow versus volume. Stated otherwise, the pulmonary flow (dV/dt) relative to the lung volume are obtained as data pairs that represent a spirometric characteristic (S). In one embodiment, the spirometric characteristic (S) is a function of only the lung volume (V) and its first time derivative (dV/dt). In another embodiment, the data pairs are produced by averaging the transthoracic impedance signal over a plurality of respiratory cycles. Further, the spirometric means selectively chooses the plurality of respiratory cycles, and in this regard optionally excludes a respiratory cycle of the plurality of respiratory cycles that includes an artifact from averaging the data pairs.

According to one embodiment, the active implantable medical device comprises one or more of the following structures and functionality:
Means for analyzing spirometric characteristic, able to infer at least one parameter representative of the patient's pulmonary condition;
Diagnostic means associated with the analyzing means, able to compare the at least one parameter of the patient's pulmonary condition, or the variation thereof in the time with a predefined reference value, and generate a diagnostic indicator as a function of the result of the comparison;
Means for identifying outstanding points of the spirometric characteristic and/or modelling the spirometric characteristic;
A priori validation means, able to determine the existence of at least one predetermined state conditions (e.g., a range of the patient's heart rate, a resting state of the patient, and a time slot) when collecting the transthoracic impedance signal, and to inhibit the operation of the means for analyzing spirometric characteristic in the absence of said state conditions;
Means of post validation, able to determine compliance of the characteristic produced through the means for analyzing spirometric characteristic with at least one predetermined criterion (e.g., the closed nature of the curve describing the spirometric characteristic on one breathing cycle, and the stability of the spirometric characteristic on a plurality of successive respiratory cycles), and to inhibit the operation of the diagnostic means in the case or non-compliance; and/or
Means for selective activation of the spirometric means operating in response to an external control signal (e.g., an external command transmitted by telemetry to the device, or a sequence of specific breathing cycles produced by the patient and detected through the spirometric means).

### BRIEF DESCRIPTION OF THE DRAWINGS

Further features, characteristics and advantages will become apparent to a person of ordinary skill in the art from the following detailed description of preferred embodiments of the present subject matter made with reference to the annexed drawings, in which:

FIG. 1 is an example of a spirometric characteristic showing the shape of the curve obtained during a full respiratory cycle;

FIG. 2 is a simplified flowchart showing the various stages of recording in the context of a respiratory monitoring;

FIG. 3 is a simplified flowchart showing the steps of recording of a forced respiration requested by a physician during a visit; and

FIG. 4 is a simplified flowchart showing the different steps of recording of a forced breathing, triggered by the patient himself.

### DETAILED DESCRIPTION

An example in accordance with a preferred embodiment of a device of the present invention will now be described with reference to the drawings.

As regards its software aspects, the invention can be implemented by suitable programming of the controlling software of a known implantable device, such as a stimulation, resynchronization, cardioversion and/or defibrillation device, including circuits and logic control for the acquisition of a signal provided by endocardial leads and/or by one or more sensors. Indeed, the present invention may be applied to implantable devices marketed by ELA Medical, Montrouge, France such as the Symphony, Rhapsody and Reply brand devices.

The present active implantable medical device comprises programmable microprocessor circuitry, including suitable memory and software instructions, to receive, form and process electrical signals collected by implanted electrodes and/or sensors, and deliver stimulation pulses to these electrodes. Software instructions to configure the device to operate in accordance with the present invention, namely to perform a spirometric measurement and analyze the resulting characteristic, can be transmitted by telemetry. Software instructions are stored in a memory of the implantable devices and executed to implement the functions of the invention as described herein. The particular adaptation of these devices to implement functions of the invention is deemed to be a design choice within the reach of a skilled-in-the-art person, and will not be described in detail.

According to one embodiment, the present active implantable medical device comprises means for measurement of transthoracic impedance, operating according to the known the principle described above. The measurement of transthoracic impedance is conventionally exploited by deriving a signal of “minute ventilation” from the peak amplitude and period of the transthoracic impedance signal, and the minute ventilation signal is used to control the pacemaker.

According to one embodiment, the parameters of the transthoracic impedance signal (e.g., peak amplitude and period) are not directly used. Rather, the signal variation of the transthoracic impedance signal over a given respiratory cycle is used. The given respiratory cycle is typically over the duration of one respiratory cycle except when prior stability respiratory cycles are used or the signals are contaminated with artifacts during the one respiratory cycle.

Spirometry is a technique based on the analysis of a respiratory characteristic collected during an isolated inspiration-expiration cycle. In principle, the collected respiratory characteristic is not averaged as opposed to the measurements of minute ventilation that give a indication only over a range of several tens of cycles or respiratory cycles. However, an average of multiple respiratory cycles may be used in a spirometry analysis for cases when some conditions are met, as will be elaborated below.

The respiratory signal being cyclical, the active implantable medical device automatically detects a breathing cycle as being defined by the signal between two successive peaks of the Z(t) curve. The registered respiratory cycle is confirmed or denied by checking, for example, that the peak amplitude at the end of the cycle is equal to ±5% of the one at the beginning of the cycle. Various techniques may be employed to eliminate cycles contaminated with artifacts, for example, due to a jump of static impedance, or interference between heart rate and breathing rate when they are close. Exemplary instances of these artifacts are described in EP 1 584 288 A1 and its counterpart U.S. Patent Application 2005/0267380, both assigned to the assignee of the present application, ELA Medical.

Impedance signals, which reflect variations of instantaneous lung volume, are recorded and stored in the memory of the device. Using the collected impedance signals, the device calculates, for each point in time t, the derivative value of the sampled signal Z(t), dZ(t)/dt, representing the instantaneous pulmonary flow dV/dt. The data pairs (or couples) of values (Z(t), dZ(t)/dt) representative of the instantaneous speed/lung volume during a given respiratory cycle, are stored in the memory of the device for further analysis.

According to one embodiment, the device records the transthoracic impedance signal Z(t) during at least one respiratory cycle when a number of prerequisite conditions are met. The recording of transthoracic impedance signal may be subjected to a number of prerequisite conditions:
During a specified time slot, when you want to record at regular intervals of a non-forced night breathing cycle, as part of the monitoring of the patient's pulmonary status;
Condition on the patient's heart rate (typically HR<70 bpm); and/or
Patient activity if the device is equipped with an activity detector (e.g., G accelerometer sensor).

Recording of the transthoracic impedance signal may also be subject to a post-validation assessment. The acquired data may be kept only if certain criteria are met, for example, when a closed curve is obtained, or when the stability of respiratory cycles is achieved in the case of non-forced respiratory cycles.

The variations of data pairs (Z (t), dZ(t)/dt) are graphically represented by a curve S in FIG. 1, presenting the flow rate variations (dV/dt) as a function of the pulmonary volume (V). The curve S is produced during a respiratory cycle including an expiratory phase and an inspiratory phase. During the expiratory phase, the flow rate increases from origin P4 rapidly and reaches its maximum after approximately 100 ms to point P1 called peak expiratory flow (PEF). The curve S then decreases more or less regularly, with a decline in the flow until there is no more volume of air expired. The point P2 at the end of expiration is called forced vital capacity (FVC). The rest of the curve corresponds to the inspiratory phase (negative flow), with the characteristic point P3 (peak inspiratory flow, DIP) and back to the origin, P4. The illustrated curve corresponds to that characteristic typically obtained for a healthy patient performing a forced expiration and inspiration.

In case of a patient with COPD, the curve presents a different shape with a much more concave shape of the expiratory phase (arc P1-P2), without substantial change in PIT and FVC values. In case of COPD, the upper respiratory paths seem to be normal up until PEF (P1), but, after the upper expiration path, the air passes through narrower paths; in case of an obstructive syndrome, these paths are partially blocked, therefore the air is expired relatively slower, resultantly the air flow is decreased giving a concave characteristic arc.

According to one embodiment, a method of automatic analysis of the spirometric characteristic is employed, for example, by monitoring the position of the characteristic points P1, P2, P3 and P4 over time, as well as the morphology of the arcs connecting the characteristic points.

According to one embodiment, the characteristic data obtained and stored in memory is simplified to keep only the remarkable or outstanding points (e.g., points P1, P4) by modelling the curvilinear arcs, for example, as four line segments A, B, C, and D represented in dotted lines in FIG. 1. Another approach for simplified data storage for analysis is to find a representative shape, for example, an elliptical, as an approximation of the spirometric curve. The analysis focuses on the direction and size of the main axes of the representative shape.

Importantly, the changes in these various parameters over time give an indication of improvement or deterioration of the patient's ventilatory status. For example:
a shift of the curve to the right over several days indicates a potential degradation of the ventilatory status;
an elevation of point P1 and a lowering of point P3 (lowering in arithmetic value, i.e., an increase in absolute value) corresponds to an increase in maximum data rates of inspiration and expiration, thus corresponding to an improvement the general ventilator state; and
a reduction in the gap between points P2 and P4 indicates a reduction in the exploited respiratory volume, that is, a deterioration of the ventilatory status.

Furthermore, a variation of plus or minus 10% of one or more of these parameters in a pre-defined period of time (e.g., a week) is considered to be an indication of a significant change in the patient's condition, and special measures may be implemented correspondingly, for example, triggering an alert, or change in clinical treatment.

In addition, in case of a proved change in the patient's respiratory state, the device may decide to stop the application of a specific pacing mode and change therapy to another. Thus, in case of dual-chamber stimulation, the device of the present invention is able to allow a spontaneous ventricular rhythm of the patient, so as to reduce the ventricular pacing rate.

In addition, a feature such as the rest rate may be turned off when a degradation of the ventilatory state is detected while in basal conditions (e.g., at night). Indeed, a way to reduce the impact of this degradation is to voluntarily increase the heart rate.

Another possible application of the present invention is the modification of the rate responsiveness function to the minute ventilation, by inhibiting the use of the ventilation sensor to allow the heart to beat at a faster rate.

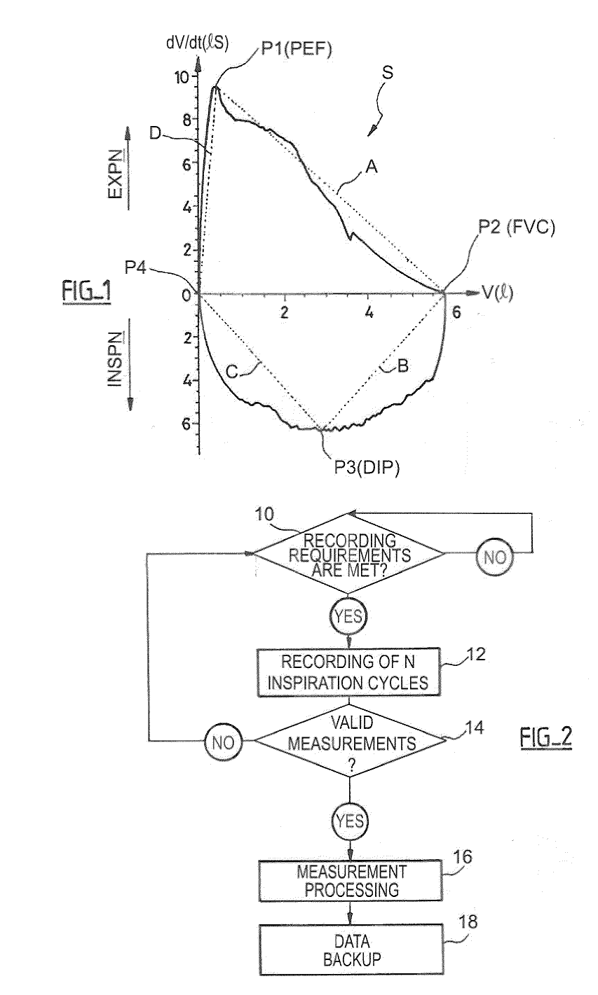

FIGS. 2, 3, and 4 illustrates several techniques for implementing the invention.

FIG. 2 illustrates a mode of implementation of the present invention in which the device performs a regular monitoring of the ventilatory function of the patient on the basis of standard (non-forced) respiratory cycles. The aim is to provide a monitoring of the current flow/volume respiratory curve to a physician during a routine visit. The data may be directly read from the memory of the implantable device or reproduced by a programmer of the physician when reading recorded data.

If a number of recording requirements are met (step 10), for example, conditions of heart rate, of time slots, of state, and of activity of the patient, the device automatically triggers data recording during one or several respiratory cycles (step 12). The recording interval is typically between one day and one week. The device checks the quality of the data for successive respiratory cycles (step 14). If the respiratory cycles are determined to be usable (e.g., by applying the post validation analysis), the data during the cycle is recorded, analyzed (step 16) and stored (step 18) in the memory of the implant for subsequent reading and interpretation by a physician.

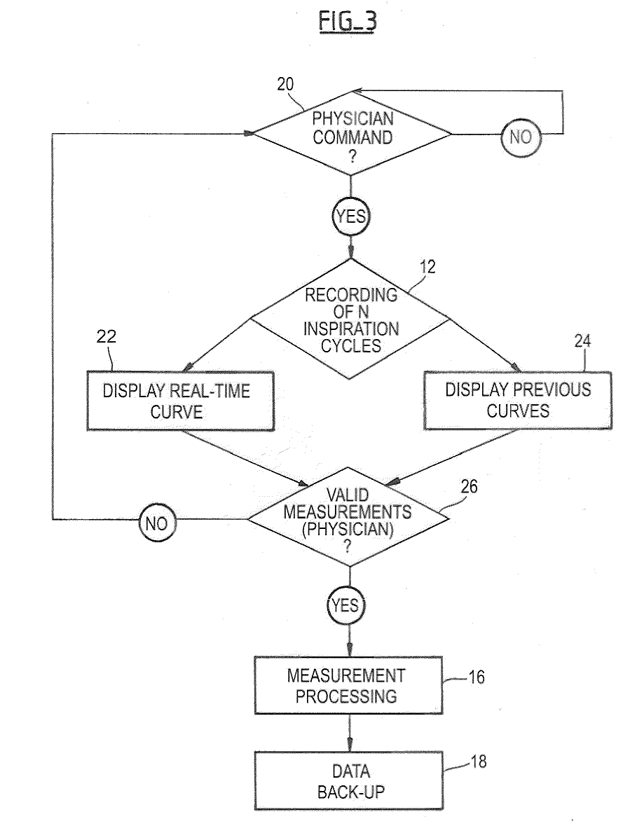

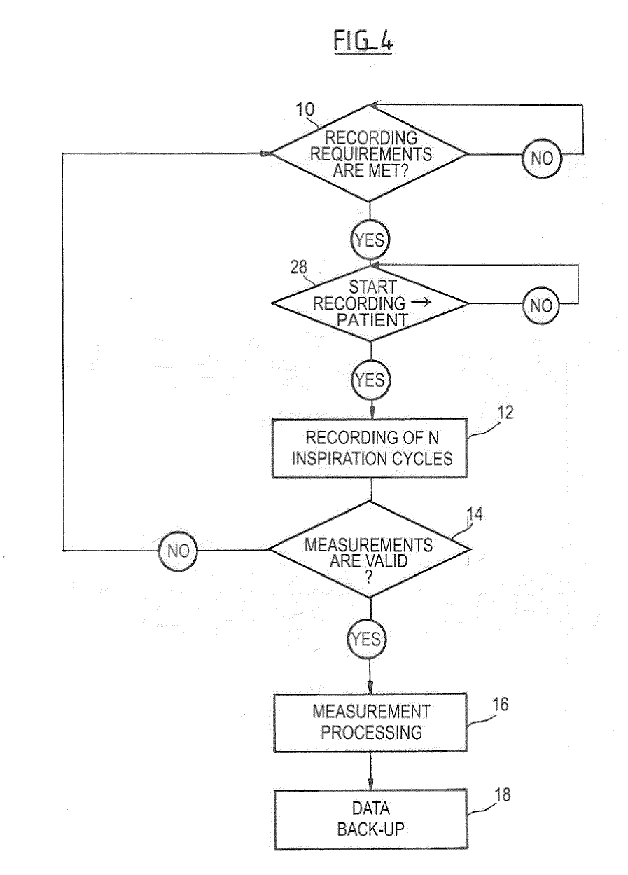

FIG. 3 illustrates another mode of implementation of the present invention, during a visit to a physician. The physician triggers data recording and asks the patient to perform a cycle of forced inspiration/expiration. The onset of the recording (step 20) may be done from the physician's programmer by telemetry. The device records respiratory data for one or more respiratory cycles (step 12), and the collected data is shown to the physician (step 22) in comparison to the past recorded records (step 24). The physician may decide to keep the recorded breathing cycle (step 26) to repeat data measurement, or to average the curve over several forced cycles. The collected data are processed (step 16) and recorded (step 18).

The advantage of the present subject mater is that spirometric data are measured (e.g. FVC, FEV) without need for a spirometer and that the measurements can be made outside of a physician's office. The physician having a programmer can read the spirometric data stored in the patient's implantable device.

FIG. 4 illustrates another mode of implementation of the present invention. The device records at regular intervals respiratory cycles, as part of a monitoring of the patient's condition. But unlike in the monitoring methods discussed above in connection with FIG. 1, the respiratory cycles are forced cycles, and an active participation of the patient is necessary.

After the recording requirements have been met (Step 10, as in FIG. 2) the recording starts (step 28). A first solution is to use the telecommunication function of the implantable device to communicate with an external device, such as a device intended for “home monitoring”. Once connected (by telemetry) to the implantable device, the external device issues a command signal to initiate a spirometric assessment and requests the patient to perform a cycle of forced inspiration/expiration. Upon confirmation of the patient, the implantable device continuously saves successive respiratory cycles (step 12) and defines the forced respiratory cycle as the breathing cycle with the highest amplitude peak-to-peak. If this cycle is compliant (step 14, as shown in FIG. 2), the collected data is analyzed (step 16) and stored (step 18).

According to another embodiment, an external device to initiate a spirometric assessment is not required. A specific respiratory sequence is defined, of which the “profile” is detected by the implantable device as a command signal and interpreted as a request or trigger for recording a forced respiratory cycle. The sequence may be a coded or fixed sequence, for example, three quick respiratory cycles at low amplitude followed by the forced inspiration/expiration cycle to be recorded and analyzed. This method has the advantage of not requiring a telemetric dialog between the implantable device and an external device. However, this technique does not allow to repeat the measure if the recording is not deemed usable, unless the implantable device is equipped to produce a confirmation (or rejection) of the validity of the measurement, for example, by a distinctive audible signal emitted by the implantable device.

One skilled in the art will appreciate that the present invention can be practiced by other than the embodiments disclosed herein, which are provided for purposes of illustration and not limitation.

I claim:

1. An active implantable medical device, comprising:
means for collecting a transthoracic impedance signal representing instantaneous lung volume variations from a patient; and
spirometric means for producing, from the transthoracic impedance signal received over at least one respiratory cycle, data pairs comprising
a lung volume (V), and
a pulmonary flow (dV/dt), represented by the derivative of said lung volume, wherein said data pairs represent a spirometric characteristic (S) corresponding to the pulmonary flow (dV/dt) relative to the lung volume.

2. The device of claim 1 further comprising means for analyzing the spirometric characteristic (S) to determine at least one parameter of the patient's pulmonary condition.

3. The device of claim 2, further comprising:
means for comparing the at least one parameter of the patient's pulmonary condition with a predetermined reference value; and
means for diagnosis for generating a diagnostic index based on the comparison.

4. The device of claim 2, wherein the means for analyzing further comprising means for searching for one or more outstanding points of the spirometric characteristic.

5. The device of claim 2, wherein the means for analyzing further comprising means for providing a linear or curvilinear model of the spirometric characteristic.

6. The device of claim 1, further comprising means for a priori validation for determining an existence of at least one predetermined state condition associated with said transthoracic impedance signal, and inhibiting the operation of the spirometric means in the absence of said at least one predetermined state condition.

7. The device of claim 6, wherein said at least one predetermined state condition further comprises a condition selected from among the group consisting of: a range of the patient's heart rate values, a resting state of the patient, and a specified time slot.

8. The device of claim 3, further comprising means for post-validating for determining whether the spirometric characteristic complies with at least one predetermined criterion, and inhibiting the operation of the means for diagnosis when said post-validating determines that the spirometric characteristic is non-compliant.

9. The device of claim 8, wherein said at least one predetermined criterion is one of a defined spirometric characteristic over one respiratory cycle having a closed curve, and a stability of the defined spirometric characteristic over a plurality of respiratory successive cycles.

10. The device of claim 1, further comprising means for selectively activating the spirometric means in response to a command signal.

11. The device of claim 10, wherein the command signal further comprises one of an external command transmitted to the device by telemetry, and a sequence of specific respiratory cycles produced by the patient and detected through the spirometric means.

12. The device of claim 1, wherein the spirometric characteristic (S) is a function of only the lung volume (V) and its first time derivative (dV/dt).

13. The device of claim 1, wherein the data pairs are produced by averaging the transthoracic impedance signal over a plurality of respiratory cycles.

14. The device of claim 13, wherein the spirometric means selectively choose the plurality of respiratory cycles.

15. The device of claim 13, wherein the spirometric means excludes a respiratory cycle of the plurality of respiratory cycles that includes an artifact from averaging the data pairs.
